(19) United States
(12) Patent Application Publication
(10) Pub. No.: US 2022/0362151 A1
(43) Pub. Date: Nov. 17, 2022

(54) LIPOSOME FORMULATION

(71) Applicant: ROSI CHARNI AS, Bønes (NO)

(72) Inventors: Rolf Kristian BERGE, Bønes (NO); Bodil BJØRNDAL, Bergen (NO)

(21) Appl. No.: 17/617,738

(22) PCT Filed: Jun. 11, 2020
(86) PCT No.: PCT/NO2020/050152

§ 371 Date: Dec. 9, 2021

(30) Foreign Application Priority Data
Jun. 11, 2019 (NO) 20190718

(51) Int. Cl. A61K 9/127; A61K 31/20

(52) U.S. Cl. CPC A61K 9/127; A61K 31/20

(57) ABSTRACT

The present invention describes liposome formulations comprising a phospholipid, cholesterol and a fatty acid compound or fatty acid containing compound. Also, various medical uses of the liposome formulation are described.

### FIELD OF THE INVENTION

The present invention relates a liposome formulation comprising a phospholipid and a fatty acid compound or fatty acid containing compound, and such liposomes for use in the prevention and/or treatment of a disorder or disease.

### BACKGROUND OF THE INVENTION

### Obesity

Obesity is a chronic disease that is highly prevalent in modern society and is associated not only with a social stigma, but also with decreased life span and numerous medical problems, including adverse psychological development, reproductive disorders such as polycystic ovarian disease, dermatological disorders such as infections, varicose veins, canthosis nigricans, and eczema, exercise intolerance, diabetes mellitus, insulin resistance, hypertension, hypercholesterolemia, cholelithiasis, osteoarthritis, orthopedic injury, thromboembolic disease, cancer, and coronary heart disease.

Existing therapies for obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery. Caloric restriction as a treatment for obesity causes catabolism of body protein stores and produces negative nitrogen balance.

Considering the high prevalence of obesity in our society and the serious consequences associated therewith as discussed above, any therapeutic drug potentially useful in reducing weight of obese persons could have a profound beneficial effect on their health. There is a need in the art for a drug that will reduce total body weight of obese subjects toward their ideal body weight without significant adverse side effects, and which also will help the obese subject to maintain the reduced weight level.

### Diabetes

Diabetes mellitus and its complications are now considered to be the third leading cause of death in Canada and the United States, trailing only cancer and cardiovascular disease. Although the acute and often lethal symptoms of diabetes can be controlled by insulin therapy, the long-term complications reduce life expectancy by as much as one third. Compared with rates of incidence in nondiabetic normal persons, diabetic patients show rates which are increased 25-fold for blindness, 17-fold for kidney disease, 5-fold for gangrene, and 2-fold for heart disease.

There are 2 major forms of diabetes mellitus. One is type I diabetes, which is also known as insulin-dependent diabetes mellitus (IDDM), and the other is type II diabetes, which is also known as noninsulin-dependent diabetes mellitus (NIDDM). Most patients with IDDM have a common pathological picture: the nearly total disappearance of insulin-producing pancreatic beta cells which results in hyperglycemia.

Considerable evidence has been accumulated showing that most IDDM is the consequence of progressive beta-cell destruction during an asymptomatic period often extending over many years. The prediabetic period can be recognized by the detection of circulating islet-cell autoantibodies and insulin autoantibodies.

### Cancer

The development of new and more effective chemotherapeutic agents for cancer treatment requires consideration of a variety of factors including cytotoxicity, tumour cell proliferation, invasion and metastasis. Conventional anticancer agents have typically been identified on the basis of their cytotoxicity alone.

Tumour progression is thought to occur when variant cells having selective growth properties arise within a tumour cell population, and one of the final stages of tumour progression is the appearance of the metastatic phenotype. During metastasis, the tumour cells invade the blood vessels, survive against circulating host immune defences, and then extravasate, implant, and grow at sites distant from the primary tumour. This ability of tumour cells to invade neighbouring tissues and to colonise other organs is among the leading causes of cancer related deaths.

The term metastasis encompasses a number of phenotypic traits which together result in the clinical problem that most often leads to death from cancer. The cells lose their adherence and restrained position within an organised tissue, move into adjacent sites, develop the capacity both to invade and to egress from blood vessels, and become capable of proliferating in unnatural locations or environments. These changes in growth patterns are accompanied by an accumulation of biochemical alterations which have the capacity to promote the metastatic process.

So far, little is known about the intrinsic mechanism involved in the metastatic cascade. It is likely that in some cases the augmented metastatic potential of certain tumour cells may be due to an increased expression of oncogenes, which normally are responsible for control of various cellular functions, including differentiation, proliferation, cell motility, and communication. Further, it has been shown that substances that modulate signal transduction pathways can inhibit the metastatic behaviour of a tumour, and it is also speculated that compounds with surface related effects, e.g. compounds which modulates the cell membranes, might be involved in the process leading to metastasis.

Cancer is a disease of inappropriate tissue accumulation. This derangement is most evident clinically when tumour tissue bulk compromises the function of vital organs

### Leukaemia

Leukemia is a broad term for heterogeneous malignant blood diseases. Leukemia arises from haematopoietic stem cells (HSCs) and/or their early progenies that have obtained mutations that turn the progenitor cells into a malignant phenotype. Blood cells are then unable to differentiate into mature cells, have uncontrolled growth, and immature leukemic cells accumulate in the bone marrow, which can be fatal if left untreated. These malignant cells can leave the bone marrow and enter the peripheral circulation and migrate to other tissues. Furthermore, the malignant cells can suppress the normal function of other non-cancerous cells. Based upon the onset and course of disease, the term leukemia is divided into acute and chronic subtypes, and these subtypes are further divided into lymphoid and myeloid subtypes. Acute leukemia has a rapid course and is often lethal if not treated within weeks or months. In contrast, chronic leukemia usually progresses slowly and has a better prognosis than acute leukemia. This master thesis will focus on acute myeloid leukemia (AML), which is the most frequent acute leukemia in adults.

Acute Myeloid Leukemia (AML) is a genetically heterogenous disease, characterized by compromised differentiation and uncontrolled clonal expansion of immature myeloid cells, primarily in the blood and bone marrow. Eventually the disease results in bone marrow failure and inadequate haematopoiesis. In AML, the myeloid stem cells develop into a type of immature white blood cell called myeloblasts that have lost their ability to mature and have an abnormal regulation of proliferation. The disease is therefore characterized by accumulation of myeloblasts cells in the bone marrow and/or blood, thus reducing the number and disrupting the function of/normal blood cells and furthermore leading to symptoms like haemorrhages, fatigue, fever and fatal infections. The malignant myeloblasts can also spread to the blood stream and from the blood and bone marrow to other parts of the body, including the skin, gums and central nervous system. If left untreated, the disease will most likely be fatal, secondary to bleeding or infection, within weeks or months after initial manifestation, reflecting the word “acute” in the name of the disease.

### Proliferative Skin Diseases

Proliferative skin diseases are widespread throughout the world and afflict millions of humans and their domesticated animals Proliferative skin diseases are characterized by keratinocyte cell proliferation, or division, and may also be associated with incomplete epidermal differentiation. Psoriasis is the most serious of the proliferative skin diseases with which this invention is concerned.

Psoriasis is a genetically determined disease of the skin characterized by two biological hallmarks First, there is a profound epidermal hyperproliferation related to accelerated and incomplete differentiation Second, there is a marked inflammation of both epidermis and dermis with an increased recruitment of T lymphocytes, and in some cases, formation of neutrophil microabcesses. Many pathologic features of psoriasis can be attributed to alterations in the growth and maturation of epidermal keratinocytes, with increased proliferation of epidermal cells, occurring within 0.2 mm of the skin's surface. Traditional investigations into the pathogenesis of psoriasis have focused on the increased proliferation and hyperplasia of the epidermis. In normal skin, the time for a cell to move from the basal layer through the granular layer is 4 to 5 weeks. In psoriatic lesions, the time is decreased sevenfold to tenfold because of a shortened cell cycle time, an increase in the absolute number of cells capable of proliferating, and an increased proportion of cells that are actually dividing. The hyperproliferative phenomenon is also expressed, although to a substantially smaller degree, in the clinically uninvolved skin of psoriatic patients.

A common form of psoriasis, psoriasis vulgaris, is characterized by well-demarcated erythematous plaques covered by thick, silvery scales. A characteristic finding is the isomorphic response (Koebner phenomenon), in which new psoriatic lesions arise at sites of cutaneous trauma. Lesions are often localized to the extensor surfaces of the extremities, and the nails and scalp are also commonly involved.

### Neurodegenerative Diseases

Neurodegenerative diseases (NDs) is characterized by progressive neuronal degeneration and death. These diseases have an increasing prevalence due to longer life expectancy and a larger share of older people in the total world population. NDs are a heterogeneous group of disorders, and often present with dementia (e.g., Alzheimer's disease, AD) or as a movement disorder (e.g., Parkinson's disease, PD). The diseases are mostly idiopathic and develop progressively and irreversibly. Current treatments focus only on reducing symptoms as there are no disease-modifying therapies.

General features of NDs include a selective loss of nerve cells and deposits of abnormal peptides in neurons or associated glial cells. The disorders are therefore often referred to as proteinopathies and include both the misfolding of proteins and their harmful aggregation intra- or extracellularly.

AD is the most common type of cognitive impairment (dementia) in all age groups. It appears mostly sporadic after the age of 65 (late-onset AD, LOAD), but 5-10% of all cases are inherited in an autosomal dominant manner typically before the age of 55 (early-onset AD, EOAD). The cause of AD is not entirely understood, but a pathological hallmark is an accumulation of amyloid-β (Aβ, plaques) mainly in the extracellular space between neurons and the formation of neurofibrillary tangles (NFT) consisting of hyperphosphorylated tau protein intracellularly in neurons. AD is also associated with the loss of neurons and synaptic function, mitochondrial abnormalities and inflammatory responses. In particular, evidence suggests that an accumulation of Aβ contributes to mitochondrial dysfunction through interaction with mitochondrial membranes and proteins. Reversely, it is also proposed that mitochondrial dysfunction in itself causes Aβ-formation and deposition, synaptic degeneration and NFT-formation. The most important risk factor for AD apart from advancing age is being a carrier of a particular variant of the apolipoprotein E gene (APOE). The gene has three alleles, ε-2, ε-3 and ε-4, where the ε-4 variant (APOE4) is associated with AD. In AD-patients, 65-80% carry at least one APOE4 allele. Carriers of two alleles have a 20-fold risk of developing AD. There is no consensus of the role of APOE in AD, but it has been shown to bind and influence the removal of A13 from the brain.

PD is the second most common type of ND after AD and the most common neurodegenerative movement disorder. The prevalence is 1-2% in people over 65, and 5-10% of the cases are familial. The main pathological features are the loss of dopaminergic neurons in the substantia nigra of the midbrain, and the accumulation of Lewy bodies mainly consisting of α-synuclein in the cytoplasm of neurons. α-synuclein is a protein with unknown functions, but is associated with presynaptic terminals and may be involved in neurotransmitter release and synaptic plasticity. As in AD, evidence indicates that mitochondrial dysfunction is a central factor in the development of PD. This may include impairment of mitochondrial biogenesis, increased reactive oxygen species (ROS) production, dysfunction in the electron transport chain (ETC) and defective mitophagy, to mention some.

Mitochondrial dysfunction plays an important role in several neurological disorders. The pathogenesis and clinical manifestations arise from the fundamental role of bioenergetics in cell biology. Eventually, cells will die if depleted of ATP. Mitochondrial injury may lead to the release of pro-apoptotic factors (e.g., cytochrome c). Many of the pathways involving mitochondrial dysfunction in AD are also prevalent in the pathogenesis of PD

Thus, the study aimed to investigate the potential effects TTA have on brain cells by using the in vitro model SH-SYSY and to compare it with the HuH-7 cell line serving as a model for liver where TTA has known effects. A secondary aim was to test different TTA-analogs in cell culture.

### Mitochondrial Dysfunction

Mitochondria power cells by generating ATP. The energy required to produce ATP is created by the highly efficient transfer of electrons down a series of carriers (Complexes I-IV) that comprise the electron transport chain (ETC). This reaction is completed by the transfer of electrons to oxygen. However, if this process does not operate properly electrons leak from members of the ETC (Complexes I and III) to oxygen increasing the formation of injurious reactive oxygen species (ROS). The low anti-oxidant capacity and high metabolic activity of neurons render these cells particularly susceptible to ROS-mediated damage. Oxidative injury resulting from mitochondrial dysfunction is a central pathological feature of neurodegenerative disorders such as Parkinson's disease, stroke, Huntington's disease, amyotrophic lateral sclerosis, Alzheimer's disease and multiple sclerosis. Treatments that reduce ROS production by improving mitochondrial function have therefore attracted considerable interest as therapeutics for these disorders, However, clinical development of neuroprotective drugs is hampered by the tremendous cost, long duration, complexity and high failure rate of human efficacy trials. Identification of an acute condition resulting from pathological processes relevant to more common neurodegenerative disorders would mitigate these problems by permitting rapid proof-of-concept to be clearly established in a small group of patients.

Mitochondrial uncoupling protein 3 is a protein that in humans is encoded by the UCP3 gene. UCP3 is a mitochondrial uncoupling protein 3, which is encoded by UCP3. The gene is located in chromosome (11q13.4) with an exon count of 7 (HGNC et al., 2016). Uncoupling protein being a supreme family of mitochondrial anion carrier. Its functions is to separate the oxidative phosphorylation from synthesis of ATP as energy which is anticipated as heat. The uncoupling proteins involves in the transferring of anions from inner mitochondrial membrane to outer mitochondrial membrane, its protein is programmed in a way to protect mitochondria from induced oxidative stress.

### Liver Diseases

### Primary Sclerosing Cholangitis

Primary sclerosing cholangitis (PSC) is a progressive liver disease, histologically characterized by inflammation and fibrosis of the bile ducts, and clinically leading to multi-focal biliary strictures and with time cirrhosis and liver failure. Patients bear a significant risk of cholangiocarcinoma and colorectal cancer, and frequently have concomitant inflammatory bowel disease and autoimmune disease manifestations. To date, no medical therapy has proven significant impact on clinical outcomes, and most patients ultimately need liver transplantation. Although the disease is relatively rare, it has reigned as the top indication for liver transplantation in Norway for decades.

The etiology of the disease is unknown and its pathophysiology incompletely understood; however, recent insights have led to increased interest as well as ongoing phase II and III trials concerning the putative therapeutic effect of nuclear and membrane receptors regulating bile acid metabolism, as well as immune modulators and compounds with effects on the gut microbiome. This far, compounds targeting bile acid toxicity demonstrate the most promising effects. Targets of interest are nuclear receptors involved in the compensatory mechanisms aiming to alleviate bile acid toxicity in cholestasis such as the farnesoid X receptor (FXR), the pregnane X receptor (PXR), and the vitamin D receptor, as well as related nuclear receptors with differing specificities, e.g. small heterodimer partner (SHP), the constitutive androstane receptor (CAR), peroxisome proliferator-activated receptor alpha (PPARα) and the glucocorticoid receptor. The selective FXR agonist OCA (6α-ethyl-chenodeoxycholic acid) has demonstrated efficacy in a dose-finding trial in PSC, but its use may be limited by pruritus as an important side effect.

PPARs (PPAR-α in particular) are critical to the regulation of hepatic transporters involved in bile homeostasis and hence logical targets for therapy in cholestatic liver diseases. PPAR agonists have anti-cholestatic effects, including enhancement of biliary phospholipid secretion and mixed micelle formation through upregulation of the multidrug resistance 3 receptor (MDR3), and inhibition of bile acid synthesis and upregulation of bile acid detoxification. In PSC, beneficial effects have been reported for the pan-PPAR agonist bezafibrate as well as the PPAR-α agonist fenofibrate.

### Primary Biliary Cholangitis

Primary biliary cirrhosis (PBC) is the most common of the autoimmune liver diseases, affecting 1:1000 women over the age of 40. The pathogenesis involves inflammation and gradual destruction of intrahepatic bile ducts leading to cholestasis, which contributes to further biliary damage in self-perpetuating cycles and may progress to cirrhosis and end-stage liver disease. Ursodeoxycholic acid (UDCA) is the standard of care and can delay histological progression and improve transplant-free survival to population level in responders. However, second-line treatment should be considered in the about 40% of patients showing biochemical non-response to UDCA as defined by validated algorithms, as non-response is associated with reduced transplant-free survival and progression to liver failure and need for liver transplantation.

Research on nuclear receptor hormones has led to the development of exciting new potential treatments including the licenced FXR agonist obeticholic acid, which however is limited due to severe pruritus as a side-effect in a substantial proportion of patients, and the pan-PPAR agonists bezafibrate (for off-label use). Promising reports have surfaced for several other substances aimed at either FXR or PPAR pathways.

### SUMMARY OF THE INVENTION

A first aspect of the present invention relates to a liposome formulation comprising; i) a phospholipid, ii) cholesterol and iii) a fatty acid compound or a fatty acid containing compound, wherein the fatty acid compound (iii) has the general formula (I):

R¹—[Z—Xᵢ]n-Y (I)
wherein R¹ is;
   a C₆-C₂₄ alkene with one or more double bonds and/or with one or more triple bonds, and/or
   a C₆-C₂₄ alkyne, or
   a C₆-C₂₄ alkyl or C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and
wherein n is an integer from 1 to 12, and
wherein i is an odd number and indicates the position relative to Y, and
wherein Xᵢ independent of each other is N, O, S, CH₂ or N—R³, and
wherein Z is CH₂ or CO or Xᵢ, and
wherein at least one Xᵢ is N or O or S, or at least one Z is CO, and
wherein R³ is CH₃ or (CH₂)₂,
wherein Y is CO—COOR₂, CH₂—COOR₂, or CH₂—R4, and wherein R4 is carboxylic acid or a derivate thereof, wherein the derivate is a carboxylic ester, a glyceride or a phospholipid
wherein R₂, if present, represents hydrogen or C1-C4 alkyl.

In a preferred embodiment, the phospholipid is selected from the group consisting of phosphatidic acid (PA), Phosphatidyletanoloamine (PE), phosphatidylcholine (PC), Phosphatidylserine (PS) or a phosphatidylinositol (PIs), preferably wherein the phospholipid is phoshatidylcholine (PC).

In a preferred embodiment, the molar ratio of phospholipid to cholesterol to fatty acid compound in the liposome is in the ratio 1.0-3 to 1 to 1 to 2, or more preferably wherein the molar ratio of phospholipid:cholesterol:fatty acid compound is about 1.8 to 1 to 1.15 or more preferably molar ratio of phospholipid:cholesterol:fatty acid compound is about 1.8 to 1 to 1.5.

In a preferred embodiment, the size of the liposomes are between 110 and 140 nm.

In a preferred embodiment, the phospholipid in compound (ii) is derived from lysophospholipids, phosphatidylserines, phosphatidylcholines, phosphatidylethanolamines, phosphatidylinositols (PI), phosphatidic acids or phosphatidylglycerols.

In a preferred embodiment, Xi is N.

In a preferred embodiment, Xi is N, and R₁ is an alkyne.

In a preferred embodiment, X1 is N and R₁ is an alkyne with one triple bond.

In a preferred embodiment, said compound is Tetradec-12-yn-1-ylglycine.

In a preferred embodiment, said compound is N-tetradecylglycine. A compound according to claim 1, wherein said compound is Tetradecylthioacetic acid.

In a preferred embodiment, the compound is 2-(tridec-12-yn-ylthio) acetic acid.

In a preferred embodiment, Xi is O.

In a preferred embodiment, X1 is O, and R₁ is an alkyne.

In a preferred embodiment, X1 is O and R₁ is an alkyne with one triple bond.

In a preferred embodiment, Xi is N—R³.

In a preferred embodiment, R3 is —CH₃.

In a preferred embodiment, R3 is —(CH₂)₂.

In a preferred embodiment, at least one Z is CO.

In a preferred embodiment, Zᵢ₌₄ is CO.

In a preferred embodiment, R1 comprises one carbon-carbon triple bond.

In a preferred embodiment, R1 comprises one carbon-carbon double bound.

In a preferred embodiment, the carbon-carbon double bond is in a cis configuration.

A second aspect of the present invention relates to a liposome formulation comprising; i) a phospholipid, ii) cholesterol and iii) a fatty acid compound for use in the prevention and/or treatment of a disorder or disease,

R¹—[Z—Xᵢ]n-Y (I)
wherein R¹ is;
   a C₆-C₂₄ alkene with one or more double bonds and/or with one or more triple bonds, and/or
   a C₆-C₂₄ alkyne, or
   a C₆-C₂₄ alkyl or C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and
wherein n is an integer from 1 to 12, and
wherein i is an odd number and indicates the position relative to Y, and
wherein Xᵢ independent of each other is N, O, S, CH₂ or N—R³, and
wherein Z is CH₂ or CO or Xᵢ, and
wherein at least one Xᵢ is N or O or S, or at least one Z is CO, and
wherein R³ is CH₃ or (CH₂)₂,
wherein Y is CO—COOR₂, CH₂—COOR₂, or CH₂—R4, and wherein R4 is carboxylic acid or a derivate thereof, wherein the derivate is a carboxylic ester, a glyceride or a phospholipid
wherein R₂, if present, represents hydrogen or C1-C4 alkyl.

In a preferred embodiment, the disorder or disease is obesity.

In a preferred embodiment, the disorder or disease is multi metabolic syndrome termed “metabolic syndrome” which is inter alia characterised by hyperinsulinemia, insulin resistance, obesity, glucose intolerance, Type 2 diabetes mellitus, dyslipidemia and/or hypertension.

In a preferred embodiment, the disorder or disease is diabetes.

In a preferred embodiment, the diabetes is type I diabetes.

In a preferred embodiment, the diabetes is type II diabetes.

In a preferred embodiment, the diabetes is a form selected from the group comprising secondary diabetes such as pancreatic, extrapancreatic/endocrine or drug-induced diabetes, or exceptional forms of diabetes such as lipoatrophic, myatonic or a diabetes caused by disturbance of insulin receptors.

In a preferred embodiment, the disorder or disease is hyperinsulinemia.

In a preferred embodiment, the disorder or disease is restenosis.

In a preferred embodiment, the formulation is for prevention or inhibition of primary or secondary neoplasms.

In a preferred embodiment, the disorder is a proliferative skin disorder.

In a preferred embodiment, the proliferative skin disorder is selected from the group comprising psoriasis, atopic dermatitis, non-specific dermatitis, primary irritant contact dermatitis, allergic contact dermatitis, lamellar ichthyosis, epidermolytic hyperkeratosis, pre-malignant sun induced keratosis, and seborrheic.

In a preferred embodiment, the proliferative skin disorder is psoriasis.

In a preferred embodiment, the disorder is an inflammatory or autoimmune disorder.

In a preferred embodiment, the inflammatory or autoimmune disorder is selected from the group comprising immune mediated disorders such as rheumatoid arthritis, systemic vasculitis, systemic lupus erythematosus, systemic sclerosis, dermatomyositis, polymyositis, various autoimmune endocrine disorders (e.g. thyroiditis and adrenalitis), various immune mediated neurological disorders (e.g. multiple sclerosis and myastenia gravis), various cardiovascular disorders (e.g. myocarditis, congestive heart failure, arteriosclerosis and stable and unstable angina, and Wegener's granulomatosis), inflammatory bowel diseases and Chron's colitis, nephritis, various inflammatory skin disorders (e.g. psoriasis, atopic dermatitis and food allergy) and acute and chronic allograft rejection after organ transplantation.

In a preferred embodiment, the disorder is a neurodegeneration disorder, or a mitochondrial dysfunction or disorders caused by hyperproliferation.

In a preferred embodiment, the neurodegenerative disorder is present in an individual with patient dementia.

In a preferred embodiment, the neurodegenerative disorder is present in an individual with Alzheimer's disease.

In a preferred embodiment, the neurodegenerative disorder is present in an individual with movement disorder.

In a preferred embodiment, the neurodegenerative disorder is present in an individual with Parkinson's disease.

In a preferred embodiment, the compound is a mitochondrial uncoupling agent for use in a mitochondrial dysfunction.

In a preferred embodiment, the compound improves the mitochondrial function.

In a preferred embodiment, the compound improves the mitochondrial uncoupling.

In a preferred embodiment, the disease/disorder related to the mitochondrial uncoupling is selected from the group consisting of metabolic diseases or disorder is selected from obesity, obesity-related complications, hypertension, cardiovascular disease, nephropathy, and neuropathy, elevated plasma glucose concentrations, type II diabetes, type I diabetes, hyperglycemia, insulin tolerance and hyperthermia.

In a preferred embodiment, the diabetes-related disease or disorder is selected from cardiovascular diseases, neurodegenerative disorders, atherosclerosis, hypertension, coronary heart diseases, cancer, alcoholic and non-alcoholic fatty liver diseases, dyslipidemia, nephropathy, retinopathy, neuropathy, diabetic heart failure, and cancer.

In a preferred embodiment, the disorder is cancer.

In a preferred embodiment, the cancer is leukemia.

In a preferred embodiment is the disease a liver disease. The liver disease can be Primary sclerosing cholangitis (PSC) or Primary biliary cirrhosis (PBC).

In a preferred embodiment is the liposome formulation treated with ultrasound or micro bubbles to increase the uptake and distribution in a tissue.

### DESCRIPTION OF THE DIAGRAMS

Embodiments of the present invention and experimental results will now be described, by way of example only, with reference to the following diagrams wherein:

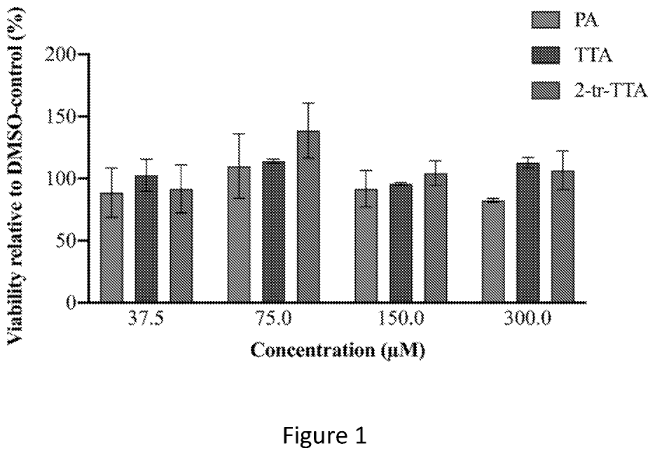

FIG. 1 shows viability of NB4 cell line treated with TTA, 2-tr-TTA and PA dissolved in DMSO after 48 hours. NB4 cells at a concentration of 0.1×10⁵ cells/mL were incubated with 37.5, 75, 150 and 300 μM of TTA, 2-tr-TTA and PA dissolved in DMSO for 48 hours. Cell viability was assessed with WST-1 assay after 4 hours of incubation, and the absorption was related to the DMSO-control. Cell viability is presented as percentage of DMSO-control (100%). The assay was performed with triplicates for each concentration of the individual SBFA and the result is presented as mean of the triplicates±SD, n=1.

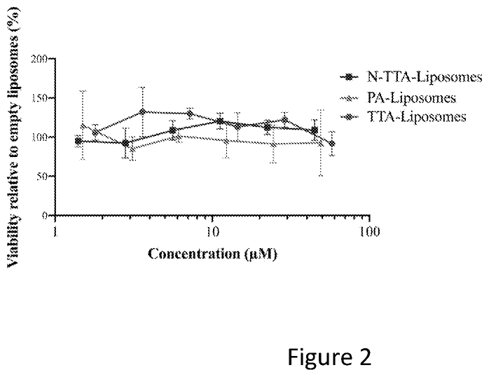

FIG. 2 shows WST-1 viability assay with MOLM-13 cell line treated with liposomes in PBS from batch 1 for 72 hours. MOLM-13 was treated with (1.8, 3.6, 7.2, 14.5 28.9 and 57.8 μM) TTA in liposomes, (1.4, 2.8, 5.5, 11.2, 22.4 and 44.8 μM) N-TTA in liposomes and (1.5, 3.1, 6.1, 12.3, 24.5 and 49.0 μM) PA in liposomes. The cell line was incubated with liposomes for 72 hours and incubated with WST-1 reagent for 4 hours. Absorbance was measured with a plate reader and absorbance of treated cells is presented as percentage of empty liposomes control (100%). All concentrations were tested in triplicates, and the experiment was repeated three times on 3 independent days (n=3). Data is presented as mean±SD.

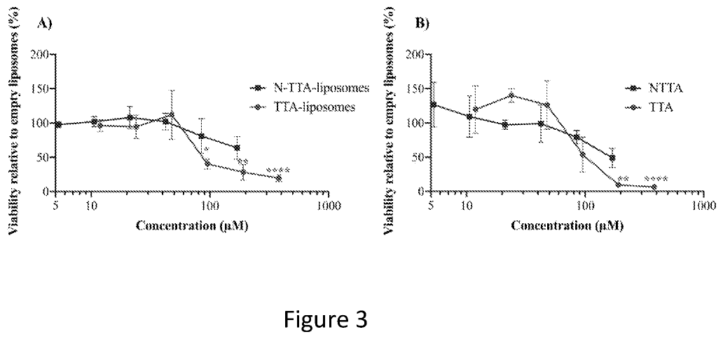

FIG. 3 shows WST-1 viability assay with AML cell lines treated with liposomes in PBS from batch 2 for 72 hours. MOLM-13 (A) and HL60 (B) was treated with (11.9, 23.9, 47.8, 95.5, 191 and 382 μM) TTA in liposomes and (5.3, 10.6, 21.2, 42.4, 84.8 and 169.6 μM) N-TTA in liposomes. The cell lines were incubated with liposomes for 72 hours and incubated with WST-1 reagent for 4 hours. Absorbance was measured with a plate reader and absorbance of treated cells is presented as percentage of empty liposomes control (set as 100%). All concentrations except HL60 with TTA-liposomes were tested in triplicates, and the experiment was repeated three times on three independent days (n=3). HL60 with TTA-liposomes was tested in triplicates on two independent days (n=2). Data is presented as mean±SD. ****=p<0.0001, **=p<0.01, *=p<0.05.

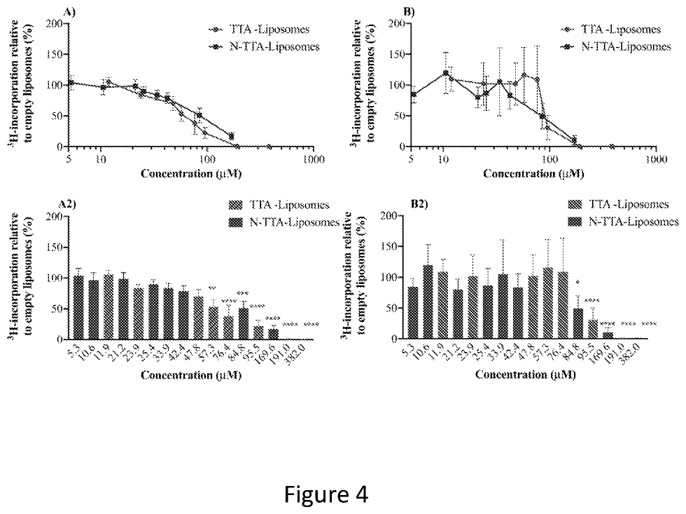

FIG. 4 shows ³H-thymidine proliferation assay with MOLM-13 (A and A2) and HL60 (B and B2). Cell lines were treated with (11.9, 23.9, 47.8, 57.3, 76.4, 95.5, 191 and 382 μM) TTA-liposomes and (5.3, 10.6, 21.2, 25.4, 33.9, 42.4, 84.8 and 169.6 μM) N-TTA-liposomes. The cell lines were incubated with liposomes for 48 hours and incubated with ³H-thymidine for 18 hours. ³H-incorporation was measured and related to empty liposome control (set as 100%) All concentrations were tested in triplicates, and the experiment was repeated three times on three independent days (n=3). Data is presented as mean±SD. In A2 and B2 ³H-incorporation is compared to empty liposome control in a two-way ANOVA, ****=p<0.0001, ***=p<0.001, **=p<0.01, *=p<0.05.

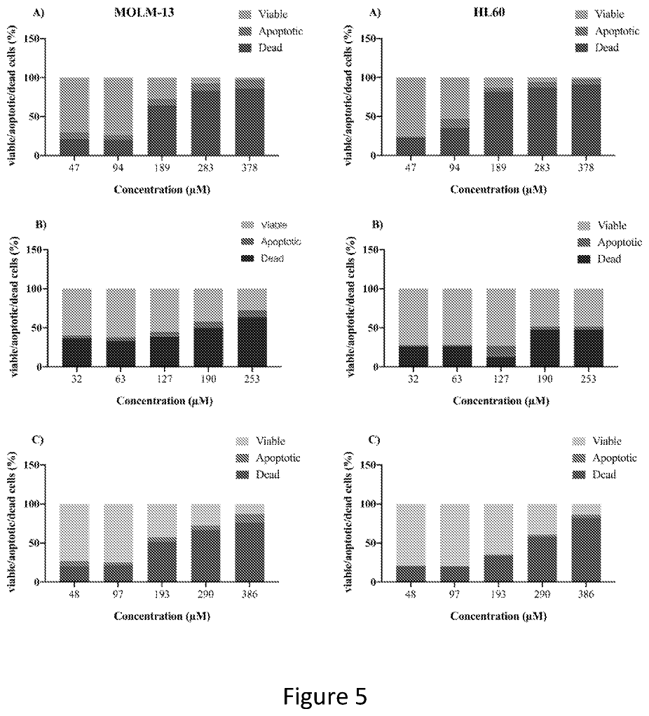

FIG. 5 shows Flow cytometric Annexin/PI apoptosis assay with PA, TTA and N-TTA in liposomes. HL60 (right) and MOLM-13 (left) (both 0.8×10⁶ cells/mL) were treated for 48 hours with (A) TTA-liposomes (47, 94, 189, 283 and 378 μM), (B) N-TTA-liposomes (32, 63, 127, 190, 253 μM) and (C) PA-liposomes (48, 97, 193, 290, 386 μM) liposomes from batch 3. SDs are removed for aesthetics reasons and presented in appendix, table x. All concentrations liposomes were tested in triplicates, and the experiment was repeated three times on three independent days.

### DESCRIPTION OF PREFERRED EMBODIMENTS OF THE INVENTION

### Experimental Section

### Example 1

### Preparation of N-tetradecylglycine (N-TTA)

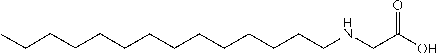 [SMILES: CCCCCCCCCCCCCCNCC(=O)O]

### Structure of N-tetradecylglycine (Termed N-TTA or TDG in the Present Application).

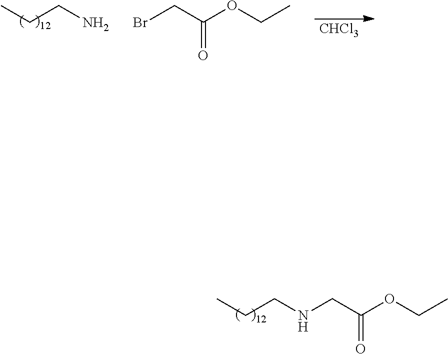

Ethyl bromoacetate (7.2 mL, 65 mmol) dissolved in chloroform (50 mL) was added dropwise to a solution of tetradecylamine (26.32 g, 123 mmol) in chloroform (250 mL) over approximately 30 minutes. After the addition was completed the reaction was stirred for an additional hour at ambient temperature.

The crude reaction mixture was reduced under reduced pressure and the product was purified by column chromatography on silica using a gradient of methanol in dichloromethane.

Yield: 14.96 g, 49.9 mmol.

¹H NMR (CDCl₃, 400 MHz): 4.17 (q, 7.1 Hz, 2H), 3.38 (s, 2H), 2.62-2.53 (m, 2H), 1.45 (m, 2H), 1.34-1.18 (m, 25H), 0.85 (t, 6.8 Hz, 3H)

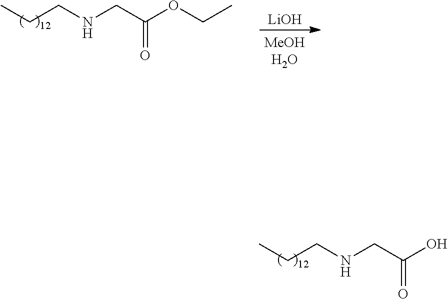

Ethyl tetradecylglycinate (19.83 g, 66.2 mmol) was dissolved in methanol (400 mL) and water (80 mL). Lithium hydroxide monohydrate (11.07 g, 264 mmol) was added and the reaction mixture was stirred over night at ambient temperature.

Formic acid (15 mL) was added dropwise to the reaction mixture and the reaction mixture was reduced under reduced pressure and the product was purified by column chromatography on reversed phase silica using a gradient of acetonitrile in water. Yield: 10.20 g (37.6 mmol).

¹H NMR (MeOH-d₄, 400 MHz): 3.49 (s, 2H), 3.03-2.90 (m, 2H), 1.73-1.63 (m, 2H), 1.43-1.23 (m, 22H), 0.90 (t, 6.8 Hz, 3H)

### Example 2—Preparation of Tetradec-12-yn-1-ylglycine hydrochloride (tr-N-TTA)

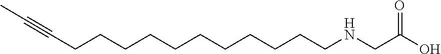 [SMILES: CC#CCCCCCCCCCCCNCC(=O)O]

Structure of tetradec-12-yn-1-ylglycine (termed tr-N-TTA or tr-TDG in the Present Application)

### tert-Butyl tetradec-12-yn-1-ylglycinate (AKB:DP-5:61-EH-1)

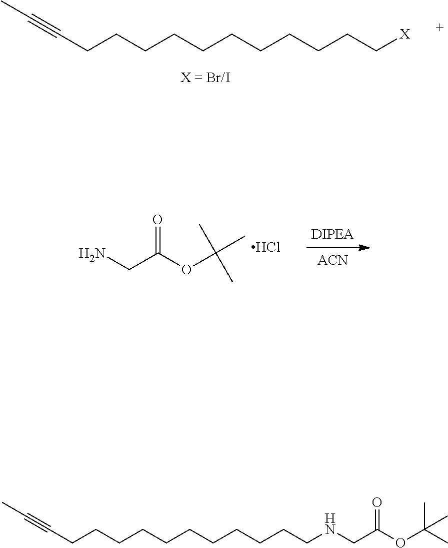

A mixture of bromo/iodotetradec-2-yne (45 g, 146 mmol) and glycine t-butyl ester hydrochloride (26.9 g, 161 mmol) in ACN, 600 ml, was added DIPEA (63.6 ml, 365 mmol) and the reaction mixture was refluxed for 4 hours. After cooling to room temperature, the mixture was concentrated under reduced pressure. Flash chromatography on silica gel eluting with heptane/EtOAc (95:5)-(70:30)-(65:35) afforded 13 g (28%) of the title compound as a yellow oil and 19 g of the starting material as bromotetradec-2-yne. ¹H NMR (400 MHz, CDCl₃) δ 3.26 (s, 2H), 2.55 (t, J=7.2, 2H), 2.16-1.92 (m, 2H), 1.75 (t, J=2.5, 3H), 1.54-1.38 (m, 14H), 1.24 (s, 13H).

### tert-Butyl tetradec-12-yn-1-ylglycinate (AKB:DP-5:61-EH-2)

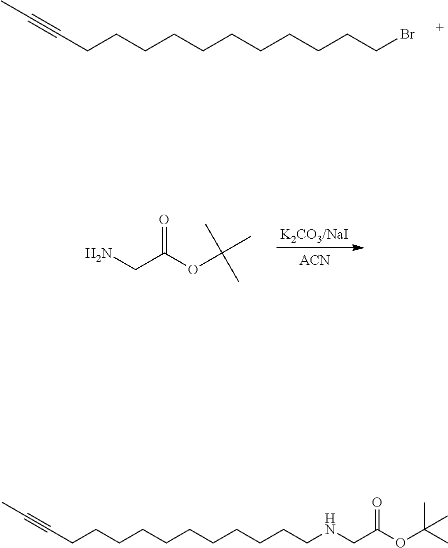

A mixture of bromotetradec-2-yne (13.6 g, 49.9 mmol) and glycine t-butyl ester hydrochloride (9.2 g, 54.9 mmol) in CAN, 200 ml, was added K₂CO₃ (17.3 g, 125 mmol) and NaI (7.5 g, 50 mmol) and refluxed overnight. The reaction mixture was cooled to room temperature, filtered and concentrated under reduced pressure. Flash chromatography on silica gel eluting with heptane/EtOAc (95:5)-(70:30)-(65:35) afforded 5.2 g (32%) of the title compound as a yellow oil and 13.8 g of the starting material.

### Tetradec-12-yn-1-ylglycine hydrochloride (EH:DP-4:82)

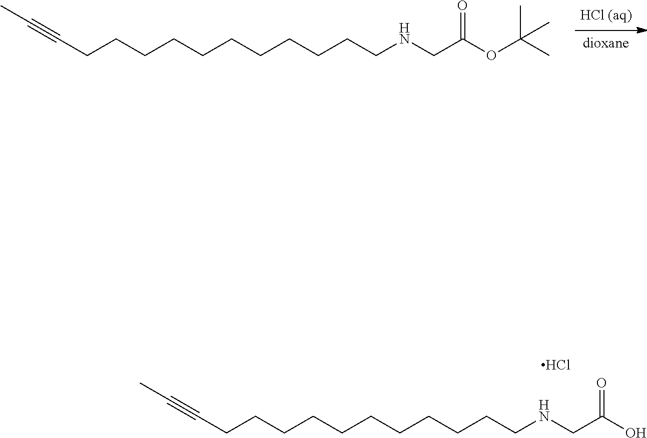

A mixture of tert-butyl tetradec-12-yn-1-ylglycinate (25.8 g, 79.7 mmol) in dioxane, 300 ml, was added 6 M HCl (80 ml) and stirred at room temperature overnight before it was stirred at 55° C. for 6 hours. The reaction mixture was cooled to room temperature and stirred overnight. Precipitated product was filtered off and washed with EtOAc, 200 ml, and dried under reduced pressure to afford 22 g (91%) as a colorless powder.

¹H NMR (400 MHz, DMSO-d6) δ 9.27 (bs, 1H), 3.80 (s, 2H), 2.96-2.78 (m, 2H), 2.57-2.43 (m, 2H), 2.19-1.99 (m, 2H), 1.71 (t, J=2.5, 3H), 1.63 (s, 2H), 1.49-1.14 (m, 14H).

¹³C NMR (101 MHz, DMSO-d6) δ 167.92, 79.28, 75.58, 46.70, 46.69, 28.89, 28.85, 28.72, 28.49 (2C), 28.44, 28.23, 25.89, 25.10, 18.01, 3.07.

MS (pos) 290 [M-HCl+Na]⁺

### Example 3—Preparation of 2-tr-TTA

### 2-(Tridec-2-yn-1-yloxy)tetrahydro-2H-pyran (AKB:TM-1:57)

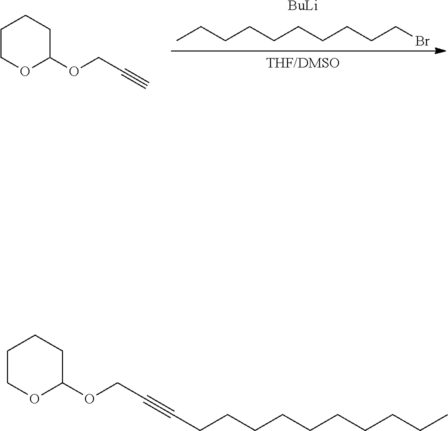

A mixture of 2-(prop-2-yn-1-yloxy)tetrahydro-2H-pyran (67.5 ml, 480 mmol) in dry THF (200 ml) was cooled to 0° C. under N₂-atmosphere before BuLi 1.6 M in hexanes (300 ml, 480 mmol) was added drop wise. 1-Bromodecane (100 ml, 483 mmol) was added followed by DMSO (1000 ml). The cooling bath was removed and the slurry was stirred for 220 minutes. The reaction mixture was cooled to 0° C. before water (250 ml) was added drop wise. Diethyl ether (600 ml) was added and the phases was separated. The organic phase was washed with a (1:1) mixture of water/brine (400 ml×4), dried (Na₂SO₄), filtered and concentrated under reduced pressure. Dry-flash chromatography on silica gel eluting with heptane-heptane:EtOAc (100:1) afforded 88.18 g (65%) of the title compound. ¹H NMR (200 MHz, CDCl₃) δ 4.80-4.77 (m, 1H), 4.40-4.02 (m, 2H), 3.95-3.70 (m, 1H), 3.55-3.44 (m, 1H), 2.31-2.06 (m, 2H), 1.99-1.05 (m, 22H), 0.85 (t, J=6.2, 3H).

### Tridec-2-yn-1-ol (AKB:TM-1:59)

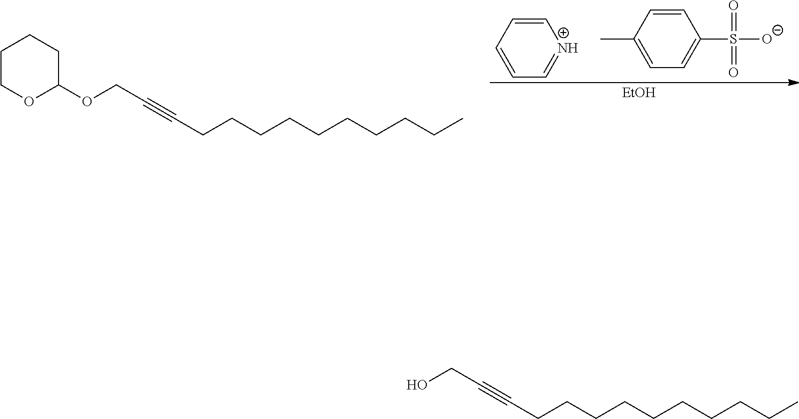 [SMILES: "EtOH" ; CCCCCCCCCCC#CCOC1CCCCO1 ; c1cc[nH+]cc1 ; Cc1ccc(S(=O)(=O)[O-])cc1 ; CCCCCCCCCCC#CCO]

A mixture of 2-(Tridec-2-yn-1-yloxy)tetrahydro-2H-pyran (AKB:TM-1:57) (85.21 g, 303.8 mmol) and PPTS (9.6 g, 38.2 mmol) in EtOH (770 ml) was stirred at 50° C. for 18 hrs and concentrated under reduced pressure. The residue was diluted with CH₂Cl₂ (500 ml) and washed with water (200 ml). The water phase was extracted with CH₂Cl₂ (500 ml). The combined organic phase was dried (Na₂SO₄), filtered and concentrated under reduced pressure. TLC showed remaining starting material. A mixture of the residue and PPTS (7.03 g, 28 mmol) in EtOH (600 ml) was stirred for 17 hrs at 50° C. and concentrated under reduced pressure. The residue was diluted with CH₂Cl₂ (500 ml) and washed with water (200 ml). The water phase was extracted with CH₂Cl₂ (500 ml). The combined organic phase was dried (Na₂SO₄), filtered and concentrated under reduced pressure. Dry-flash chromatography on silica gel eluting with heptane:EtOAc (100:1)-(95:5)-(80:20) afforded 46.06 g (77%) of the title compound as a colorless waxy solid. ¹H NMR (200 MHz, CDCl₃) δ 4.27-4.21 (m, 2H), 2.23-2.15 (m, 2H), 1.65-1.25 (m, 17H), 0.90-0.82 (m, 3H).

### Tridec-12-yn-1-ol (AKB:TM-1:63)

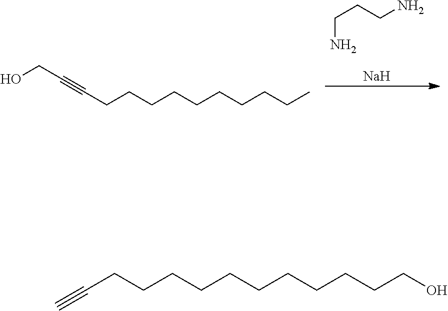

Sodium hydride 60% dispersion in mineral oil (38.82 g, 970.5 mmol) in 1,3-diaminopropane (500 ml) was stirred at 70° C. for 1 hr. The mixture was cooled to room temperature before a solution of tridec-2-yn-1-ol (AKB:TM-1:59) (23.95 g, 122 mmol) in 1,3-diaminopropane (250 ml). The reaction mixture was stirred at 55° C. under N₂-atmosphere for 20 hrs. The mixture was cooled in an ice-bath and water 1000 ml was added. The mixture was extracted with diethyl ether (500 ml×4), washed with 1 M HCl (500 ml), water (500 ml) and brine (300 ml), dried Na₂SO₄, filtered and concentrated under reduced pressure. Dry-flash chromatography on silica gel eluting with heptane-heptane:EtOAc (95:5)-(80:20) afforded 19.76 g (83%) of the title compound. ¹H NMR (200 MHz, CDCl₃) δ 3.62 (dd, J=11.7, 6.4, 2H), 2.16 (td, J=6.9, 2.6, 2H), 1.91 (t, J=2.6, 1H), 1.70-1.05 (m, 18H).

### 13-Bromotridec-1-yne (AKB:TM-1:65)

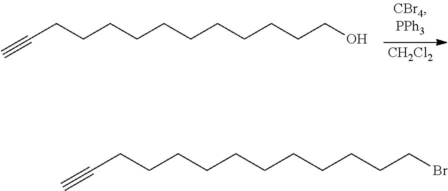

A solution of tridec-12-yn-1-ol (35.27 g, 180 mmol) in dry CH₂Cl₂ (700 ml) was cooled to 0° C. before addition of triphenylphosphine (51.86 g, 197.7 mmol) followed by tetrabromomethane (65.62 g, 197.9 mmol). The reaction mixture was stirred at 0° C. under N₂-atmosphere for 2 hrs. Silica gel was added and the mixture was concentrated under reduced pressure. Dry-flash chromatography on silica gel eluting with heptane afforded 45.55 g (98%) of the title compound as a colorless liquid which solidified upon storage in the freezer. ¹H NMR (200 MHz, CDCl₃) δ 3.38 (t, J=6.8, 2H), 2.16 (td, J=6.9, 2.6, 2H), 1.91 (t, J=2.6, 1H), 1.81 (dd, J=14.7, 6.8, 2H), 1.62-1.11 (m, 16H).

### 14-Bromotetradec-2-yne (AKB:TM-1:67)

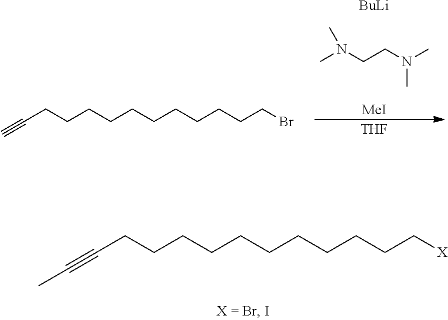

A solution of 13-bromotridec-1-yne (AKB:TM-1:65) (44.68 g, 172.4 mmol) in dry THF (500 ml) was cooled to −10° C. under N₂-atmosphere before BuLi 1.6 M in hexanes (118.5 ml, 189.6 mmol) was added drop wise. The reaction mixture was stirred for 10 minutes before TMEDA (56.5 ml, 376.3 mmol) was added drop wise followed by drop wise addition of methyl iodide (57 ml, 915.6 mmol). A white solid precipitated and extra THF was added in order to stir the reaction mixture. The cooling bath was removed and the reaction mixture was stirred for 18 hrs. Water (500 ml) was added and the phases were separated. The water phase was extracted with diethyl ether (500 ml×2), washed with 1 M HCl (aq) (300 ml), dried (Na₂SO₄), filtered and concentrated under reduced pressure to afford the crude title compound as a mixture of the bromo- and iodo-compound.

### 2-(Tetradec-12-yn-1-ylthio)acetic acid (AKB:TM-1:69/GH:DP-3:42)

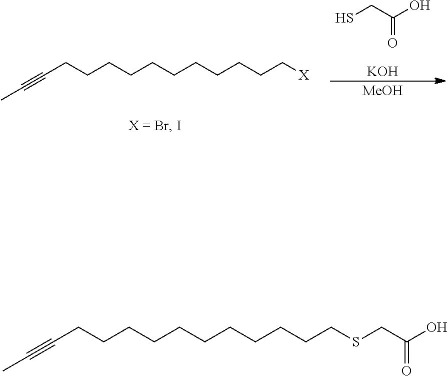

Potassium hydroxide (25.05 g, 446 mmol) was dissolved in MeOH (270 ml) before a solution of 2-mercaptoacetic acid (14 ml, 201.4 mmol) in MeOH (270 ml) was added drop wise. The reaction mixture was stirred for 10 minutes before 14-bromotetradec-2-yne/14-iodotetradec-2-yne (AKB:TM-1:67) (49.74 g) was added drop wise. The 14-bromotetradec-2-yne/14-iodotetradec-2-yne flask was washed out with MeOH (100 ml). The reaction mixture was stirred at 50° C. for 16 hrs, cooled to 0° C. and 1 M and 6 M HCl (aq) was added to pH 1-2 and water 250 ml was added. The mixture was extracted with diethyl ether (1000 ml×2), dried (MgSO₄), filtered and concentrated under reduced pressure. Recrystallization from heptane/EtOAc afforded 22.9 g of the title compound as a light yellow solid. The mother liquor was dissolved in diethyl ether and precipitated with heptane to afford another 10.8 g of the title compound. Total yield 33.7 g (69% from 13-bromotridec-1-yne). ¹H NMR (400 MHz, CDCl₃) δ 11.58 (s, 1H), 3.18 (s, 2H), 2.65-2.51 (m, 2H), 2.06-2.02 (m, 2H), 1.71 (t, J=2.6, 3H), 1.61-1.48 (m, 2H), 1.42-1.36 (m, 2H), 1.25 (d, J=36.2, 14H). MS (neg): 283 [M-H]⁻

### Example 4—Preparation of 1-tr-TTA

1-tr-TTA was obtained in a similar process as described in example 3, but the third last step can be omitted.

### Example 5—Preparation of Liposomes

Liposomes (lipid vesicles) were prepared with a technique called lipid extrusion. The basic principle of this method is to press a lipid suspension through a polycarbonate filter with defined pore size at a temperature above the lipids transition temperature. Before the extrusion process, a thin lipid film (also called lipid cakes) is produced. When the lipid film is rehydrated, the stacks of crystalline bilayers within the lipid film swell. During agitation, the lipids sheets self-assembly into large multilamellar vesicles (MLV). With decreasing pore size, the extrusion pressure increases. At higher pressure, the vesicles are broken down and the phospholipid bilayer is reorganized, resulting in unilammelar vesicles.

Hydrogenated egg phosphatidylcholine (HEPC), cholesterol (CHO) and fatty acid compound (PA, N-TTA and TTA) were weighed out separately and dissolved in chloroform. N-TTA is not soluble in chloroform alone and was therefore dissolved in a 1:1 mixture of methanol and chloroform. The dissolved lipids were mixed in a molar ratio of 1.81 HEPC:1CHO and 0.5-2 PA/N-TTA/TTA in a 100 or 250 mL Duran round bottom flasks. Next, a thin lipid film was made by slowly evaporating the solvents by using Laborota 4000 rotary evaporator at light vacuum, 200 mbar, and 60 rpm for 1.5-2.5 hours depending on the volume of solvents. To ensure a solvent-free lipid film, full vacuum (0 mbar) was applied the last 30 minutes.

The lipid film was then rehydrated in 70° C. PBS 70° C. by alternating between a Vortex Genie and 70° C. water bath. The lipid suspension was protected with plastic film until large unilammelar vesicles (LUVs) were prepared with a mini extruder set from Avanti® Polar lipids. The mini extruder was placed on a DRI-BLOCK® heating block, ensuring approximately 70° C. through the extruding process, as this temperature is above the lipids transition temperature. In the extruding process, the hydrated lipid film was passed through Whatman® Nucleopore® Track-Etched polycarbonate membranes with decreasing pore size. Firstly, the suspension was passed 11 times through a 400 nm pore size membrane. Secondly, the suspension was pressed 11 times through a 200 nm pore size membrane, and lastly the suspension was passed 22 times through a 100 nm pore size membrane. The membranes and Avanti® filter supports were regularly replaced with new, intact membranes during this process. This resulted in liposomes between 110-140 nm. Finally, liposomes were stored in sterile Eppendorf tubes protected from light at 4° C.

The liposomes were stored for maximum 6 weeks, and the liposome solution was always mixed before use in experiments and analysis. Empty liposomes were prepared similarly as liposomes with PA/N-TTA/TTA, except these FAs were not added. Round-bottom flasks and PBS was autoclaved before use. Clean gloves were always used in preparation of lipids and in handling of the equipment in order to avoid contamination with lipids from the human skin and environment. When handling organic solvents, glass pipettes were always used.

The quantity of PA, TTA and N-TTA in the liposomes was estimated with GLC-FID.

After preparation of B SFAs, the B SFAs were investigated for their cytotoxic potential on NB4, MOLM-13 and HL60. The cytotoxic effect was investigated by three different methods, i) WST-1 viability assay, ii) ³H-thymidine proliferation assay and iii) Apoptosis assay performed with flow cytometry.

### Example 6—Assessment of Cell Viability by WST-1 Assay

As a preliminary test, NB4 was exposed to TTA, 2-tr-TTA and PA dissolved in DMSO in concentrations between 37.5 to 300 μM for 48 hours. WST-1 assay with TTA, PA and 2-tr-TTA dissolved in DMSO was only performed once on NB4, and was not tested further in this project due to the lack of significant antiproliferative effect (data not shown). It was decided to not try higher concentrations of TTA PA and 2-tr-TTA dissolved in DMSO because of DMSO's cellular toxicity. In this project, one aim was to compare TTA with N-TTA and 2-tr-N-TTA. Because N-TTA and 2-trN-TTA were seemingly insoluble in DMSO, it was considered unreasonable to continue with DMSO as a solvent. Compared to DMSO-control there was no significant decrease in cell viability after treatment with B SFAs dissolved in DMSO (p>0.05). As presented in FIG. 1, 2-tr-TTA seemed to have a stimulating effect, especially at a concentration of 75 μM. More interestingly, PA seemed to decrease metabolic activity more than TTA.

Liposomes containing TTA, N-TTA and PA was prepared as described above, and the potential anti-proliferative effect the investigated with WST-1 assay as described above. FIG. 2 show viability after treatment with liposomes from batch 1. FIG. 3 displays viability after treatment with liposomes from batch 2. In both experiments, cell lines were incubated with liposomes for 72 hours, and incubated with WST-1 reagent for 4 hours. Absorption, obtained by plate reader, was related to absorption from cells treated with empty liposomes and presented as percent viability of empty liposomes control. Liposomes from batch 1 showed no significant inhibitory effect on metabolic activity on MOLM-13 (p>0.05).

Batch 2 of liposomes was prepared with a higher concentration of BSFAs than batch one due to lack of inhibitory effect on metabolic activity in batch one measured with WST-1. Results from WST-1 assay is presented in FIG. 3. Compared to empty liposomes (set as 100%), liposomes from batch 2, especially liposomes with TTA, show a considerable reduction of viability. For liposomes with TTA, viability seemed to drop with higher concentrations than 47.8 μM. N-TTA in liposomes seemed to a lesser extent than TTA-liposomes to decrease viability in MOLM-13 and HL60. N-TTA-liposomes did not give a significant decrease in viability (p>0.05). For both MOLM-13 and HL60, 382 μM (p<0.0001), 191 μM (p<0.01) and 95.5 μM (p<0.05, only in MOLM-13) significantly decreased viability compared to empty liposomes. Simultaneously, cell lines were exposed to the same concentration with PBS as the cells in the experiment with liposomes were. 10% PBS had no significant inhibitory effect on the cells.

The anti-proliferative effect of liposomes with N-TTA and TTA was studied with ³H-thymidine incorporation assay on HL60 and MOLM-13, and the anti-leukaemic effect was compared to WST-1 assay with liposomes from the same batch. The cells were treated with TTA-liposomes in concentrations from 11.9-382.0 μM, and N-TTA-liposomes in concentrations from 5.3-169.6 μM. The cell lines were incubated with the liposomes for 48 hours, and further incubated with ³H-thymidine solution for 18 hours. The ³H-thymidine incorporation was compared to ³H-thymidine incorporation in empty liposomes (set as 100%). FIG. 4 shows mean proliferation compared to empty liposome control±SD. A dose-dependent anti-proliferative effect was seen in both cell lines after treatment with both TTA-liposomes and N-TTA-liposomes. As demonstrated with the large SD in FIG. 4B, results varied greatly for HL60. The results from MOLM-13 were considerably less wide-ranging. Compared to control with empty liposomes, there was observed a significant decrease in cell proliferation in HL60 and MOLM-13 treated with 382, 191 and 95.5 μM of TTA-liposomes (p<0.0001). In addition, proliferation was significantly decreased in MOLM-13 with 76.4 μM (p<0.0001) and 57.3 μM (p<0.05) of TTA-liposomes. Whereas, N-TTA-liposomes significantly decreased cell proliferation after treatment with 169.6 μM (p<0.0001) in both cell lines and 84.8 μM (p<0.05, HL60 and p<0.001, MOLM-13).

In order to quantify apoptotic, dead, and viable cells after treatment with TTA-, N-TTA- and PA-liposomes, annexin/PI apoptosis assay was performed on MOLM-13 and HL60 with flow cytometry after 48 hours of incubation with liposomes from batch 3.

HL60 and MOLM-13 was treated with (47, 94, 189, 283 and 378 μM) TTA-liposomes, (48, 97, 193, 290, 386 μM) PA-liposomes and (32, 63, 127, 190, 253 μM) N-TTA-liposomes.

The results are shown in FIG. 5. The results show high degree of apoptosis among the cells after treatment with liposomes with TTA, especially in concentrations higher than 100 μM.

1.-53. (canceled)

54. A liposome formulation comprising; i) a phospholipid, ii) cholesterol and iii) a fatty acid compound or a fatty acid containing compound, wherein the fatty acid compound (iii) has the general formula (I):
R¹—[Z—Xᵢ]n-Y (I)
wherein 10 is;
a C₆-C₂₄ alkene with one or more double bonds and/or with one or more triple bonds, and/or a C₆-C₂₄ alkyne, or
a C₆-C₂₄ alkyl or C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and
wherein n is an integer from 1 to 12, and
wherein i is an odd number and indicates the position relative to Y, and
wherein Xᵢ independent of each other is N, O, S, CH₂ or N—R³, and
wherein Z is CH₂ or CO or Xᵢ, and
wherein at least one Xᵢ is N or O or S, or at least one Z is CO, and
wherein R³ is CH₃ or (CH₂)₂,
wherein Y is CO—COOR₂, CH₂—COOR₂, or CH₂-R₄, and wherein R₄ is carboxylic acid or a derivate thereof, wherein the derivate is a carboxylic ester, a glyceride or a phospholipid wherein R₂, if present, represents hydrogen or C1-C4 alkyl.

55. The liposome formulation according to claim 54, wherein the phospholipid is selected from the group consisting of phosphatidic acid (PA), phosphatidyletanoleamine (PE), phosphatidylcholine (PC), phosphatidylserine (PS), and a phosphatidylinositol (PIs).

56. The liposome formulation according to claim 54, wherein the molar ratio of phospholipid to cholesterol to fatty acid compound in the liposome is about 1-3 to 1 to 1-2.

57. The liposome formulation according to claim 56, wherein the molar ratio of phospholipid to cholesterol to fatty acid compound in the liposome is about 1.8 to 1 to 1.15.

58. The liposome formulation according to claim 56, wherein the molar ratio of phospholipid to cholesterol to fatty acid compound in the liposome is about 1.8 to 1 to 1.5.

59. The liposome formulation according to claim 54, wherein the size of the liposomes is between 110 and 140 nm.

60. The liposome formulation according to claim 54, wherein Xi is N.

61. The liposome formulation according to claim 54, wherein said compound is Tetradec-12-yn-1-ylglycine.

62. The liposome formulation according to claim 54, wherein said compound is N-tetradecylglycine.

63. The liposome formulation according to claim 54, wherein the compound is 2-(tridec-12-yn-ylthio) acetic acid.

64. A liposome formulation comprising; i) a phospholipid and ii) cholesterol and iii) a fatty acid compound or a fatty acid containing compound for use in the prevention and/or treatment of a disorder or disease, wherein the fatty acid compound (iii) has the general formula (I):
R¹—[Z—Xᵢ]n-Y (I)
wherein R¹ is;
a C₆-C₂₄ alkene with one or more double bonds and/or with one or more triple bonds, and/or a C₆-C₂₄ alkyne, or
a C₆-C₂₄ alkyl or C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and
wherein n is an integer from 1 to 12, and
wherein i is an odd number and indicates the position relative to Y, and
wherein Xᵢ independent of each other is N, O, S, CH₂ or N—R³, and
wherein Z is CH₂ or CO or Xᵢ, and
wherein at least one Xᵢ is N or O or S, or at least one Z is CO, and
wherein R³ is CH₃ or (CH₂)₂,
wherein Y is CO—COOR₂, CH₂—COOR₂, or CH₂—R4, and wherein R4 is carboxylic acid or a derivate thereof, wherein the derivate is a carboxylic ester, a glyceride or a phospholipid wherein R₂, if present, represents hydrogen or C1-C4 alkyl.

65. The liposome formulation for use in accordance with claim 64, wherein the disorder or disease is obesity, or wherein the disorder or disease is multi metabolic syndrome termed “metabolic syndrome” which is inter alia characterised by hyperinsulinemia, insulin resistance, obesity, glucose intolerance, Type 2 diabetes mellitus, dyslipidemia and/or hypertension, or wherein the disorder or disease is diabetes, or hyperinsulinemia or restenosis, or the disorder is a proliferative skin disorder, or an inflammatory or autoimmune disorder, or wherein the disorder is a neurodegeneration disorder or a mitochondrial dysfunction or disorders caused by hyperproliferation.

66. The liposome formulation for use according to claim 65, wherein the neurodegenerative disorder is present in an individual with patient dementia.

67. The liposome formulation for use according to claim 65 wherein the neurodegenerative disorder is present in an individual with Alzheimer's disease.

68. The liposome formulation for use according to claim 65, wherein the neurodegenerative disorder is present in an individual with movement disorder.

69. The liposome formulation for use according to claim 65, wherein the neurodegenerative disorder is present in an individual with Parkinson's disease.

70. The liposome formulation for use according to claim 65, wherein the compound is a mitochondrial uncoupling agent for use in a mitochondrial dysfunction.

71. The liposome formulation according to claim 65, wherein the disorder is cancer.

72. The liposome formulation for use according to claim 65, wherein the disease is a liver disease.

73. The liposome formulation for use according to claim 65, wherein the liposome formulation is treated with ultrasound or micro bubbles to increase the uptake and distribution in a tissue.
